(19) United States
(12) Patent Application Publication
(10) Pub. No.: US 2016/0346606 A1
(43) Pub. Date: Dec. 1, 2016

(54) SWING DATA COMPRESSION METHOD, SWING DATA COMPRESSION APPARATUS, SWING ANALYSIS APPARATUS, AND SWING DATA COMPRESSION PROGRAM

(71) Applicant: SEIKO EPSON CORPORATION, Tokyo (JP)

(72) Inventors: Shoichi NAKAJO, Matsumoto-shi (JP); Hisao TANAKA, Shiojiri-shi (JP)

(73) Assignee: SEIKO EPSON CORPORATION, Tokyo (JP)

(21) Appl. No.: 15/114,619

(22) PCT Filed: Mar. 24, 2015
(86) PCT No.: PCT/JP2015/001672

§ 371 Date: Jul. 27, 2016

(30) Foreign Application Priority Data
Mar. 27, 2014 (JP) 2014-066074

(51) Int. Cl. A63B 24/00; A61B 5/00; A63B 60/46; A61B 5/11; G09B 19/00; A63B 69/36

(52) U.S. Cl. CPC A63B 24/0006; G09B 19/0038; A63B 69/36; A63B 60/46; A61B 5/1116; A61B 5/6895; A63B 2071/0647

(57) ABSTRACT

To provide a swing data compression method, a swing data compression apparatus, a swing analysis apparatus, and a swing data compression program in which it is possible to reduce the quantity of swing data that is preserved while swing analysis accuracy is maintained. A swing data compression apparatus includes a storage unit that stores swing data which is output from an inertial sensor mounted on a detection target; an analysis unit that analyzes a swing of the detection target using the swing data stored in the storage unit; and a compression unit that compresses the swing data according to a compression rate which is set based on a result of swing analysis.

### TECHNICAL FIELD

The present invention relates to a swing data compression method, a swing data compression apparatus, a swing analysis apparatus, and a swing data compression program.

### BACKGROUND ART

An apparatus is known in which an inertial sensor-equipped sensor unit is mounted on a golf club, outputs from the inertial sensor are transmitted to and analyzed by an analysis apparatus (personal computer), thereby visualizing a swing (refer to PTL 1).

### CITATION LIST

### Patent Literature

PTL 1: JP-A-2008-73210

### SUMMARY OF INVENTION

### Technical Problem

The inertial sensor collects swing data at a high sampling rate in order to analyze fast-moving golf swing, and thus the quantity of data to be transmitted to the analysis apparatus increases. In addition, if, for example, an inertial sensor element which has multiple axes, such as three axes orthogonal each other, is used as the inertial sensor, the quantity of data, which is acquired by multiplying the number of sensor elements, is collected. Further, if a plurality of sensors, such as an acceleration sensor and a gyro sensor, are used as the inertial sensor, the quantity of data further increases. Therefore, the load of storage of data increases in the analysis apparatus.

An object of some aspects of the invention is to provide a swing data compression method, a swing data compression apparatus, a swing analysis apparatus, and a swing data compression program in which it is possible to reduce the quantity of swing data that is preserved while swing analysis accuracy is maintained.

### Solution to Problem

(1) An aspect of the invention relates to a swing data compression method including: storing swing data which is output from an inertial sensor mounted on a detection target; analyzing a swing of the detection target using the stored swing data; and compressing the swing data according to a compression rate which is set based on a result of swing analysis.

According to the aspect of the invention, a compression rate (non-compression size/compression size) is set based on the result of swing analysis, for example, the characteristics of the swing such as swing speed or the stability of a swing. The swing data is compressed according to the compression rate. Therefore, the quantity of swing data decreases, with the result that it is possible to reduce the memory capacity of a final storage side, and thus it is possible to reduce the load (transfer time or the like) which is required to transfer the swing data. In addition, the compression rate is set based on the characteristics of a swing and it is possible to remove unnecessary data which is less important for the swing analysis, and thus it is possible to maintain the swing analysis accuracy.

(2) In the aspect of the invention, the swing data compression method may further include: analyzing swing speed of the detection target; and making setting such that the compression rate of the swing data in which the swing speed is slow is higher than the compression rate of the swing data in which the swing speed is rapid. Since the swing data, in which the swing speed is slow, has a small amount of change per unit time, the swing analysis accuracy is less affected when data is omitted by increasing the compression rate.

(3) In the aspect of the invention, the swing data compression method may further include: compressing the swing data by thinning the swing data of one swing which continues in time sequence according to the compression rate. When the data is thinned, the quantity of data per unit time decreases. However, since the swing data, in which the swing speed is slow, has a small amount of change per unit time, the swing analysis accuracy is less affected.

(4) In the aspect of the invention, the swing data compression method may further include: analyzing at least one of posture and position of the detection target in multiple swings; and making setting such that the compression rate of the swing data, in which variation in at least one of the posture and the position of the detection target in each swing is large, is higher than the compression rate of the swing data in which variation in at least one of the posture and the position of the detection target in each swing is small. The swing data, in which the variation in at least one of the posture and the position of the detection target is large, is data which has poor stability. If the swing varies, data indicative of resolution which stops in the range of the variation does not contribute to the analysis accuracy, and thus it is possible to increase the compression rate.

(5) In the aspect of the invention, the swing data compression method may further include compressing the swing data by discarding lower bits of a plurality of bits which form one swing data set according to the compression rate. It is possible to remove the unnecessary data indicative of the resolution which stops in the range of the variation by discarding the lower bits.

(6) In the aspect of the invention, the swing data compression method may further include: analyzing a standard deviation a of at least one of the posture and the position of the detection target in the multiple swings; and making setting such that the compression rate of the swing data, in which a range of ±3σ or ±4σ is wide, is higher than the compression rate of the swing data, in which the range of ±3σ or ±4σ is narrow. As the range of ±3σ or ±4σ, in which there is a high possibility that the population of the data is approximately covered, is narrow, the resolution is high. As the range of ±3σ or ±4σ is wide, the resolution is low. Accordingly, high resolution is not necessary for the swing data in which the range of ±3σ or ±4σ is wide, and thus the analysis accuracy does not deteriorate when the compression rate increases.

(7) Another aspect of the invention relates to a swing data compression apparatus including: a storage unit that stores swing data which is output from an inertial sensor mounted on a detection target; an analysis unit that analyzes swing of the detection target using the swing data stored in the storage unit; and a compression unit that compresses the swing data according to a compression rate which is set based on a result of swing analysis. According to the another aspect of the invention, it is possible to preferably implement the compression method according to the first aspect.

(8) Still another aspect of the invention relates to a swing analysis apparatus including: a storage unit that stores swing data which is output from an inertial sensor mounted on a detection target; an analysis unit that analyzes a swing of the detection target using the swing data stored in the storage unit; and a compression unit that compresses the swing data according to a compression rate which is set based on a result of swing analysis. According to the still another aspect of the invention, the compression rate is set based on the characteristics of the swing and it is possible to remove unnecessary data which is less important for the swing analysis, and thus it is possible to maintain the swing analysis accuracy.

(9) In the still another aspect of the invention, the swing analysis apparatus may further include: a sampling rate setting unit that sets a sampling rate such that the inertial sensor performs sampling on the swing data based on the swing analysis. If the sampling rate is lowered, the quantity of swing data decreases, thereby being equivalent to data compression in a stage of collection. Accordingly, after the sampling rate is changed, a data compression operation is not necessary.

(10) Yet another aspect of the invention relates to a swing data compression program causing a computer to perform: a procedure for storing swing data which is output from an inertial sensor mounted on a detection target; a procedure for analyzing a swing of the detection target using the stored swing data; and a procedure for compressing the swing data according to a compression rate which is set based on a result of swing analysis. According to the yet another aspect of the invention, it is possible to implement the compression method, the data compression apparatus, and the swing analysis apparatus according to the aspects of the invention described above by software.

### BRIEF DESCRIPTION OF DRAWINGS

FIG. 1 is a general diagram illustrating a swing analysis system according to an embodiment of the invention.

FIG. 2 is a block diagram illustrating a swing analysis apparatus according to the embodiment of the invention.

FIG. 3 is a block diagram illustrating an arithmetic processing circuit illustrated in FIG. 2.

FIG. 4 is a view illustrating a golf swing trajectory.

FIG. 5 is a flowchart illustrating the outline of a swing data compression operation.

FIG. 6 is a view illustrating the face side of a club head, which is used as a reference when a compression rate is set.

FIG. 7 is a block diagram illustrating an example of a posture computing unit illustrated in FIG. 3.

FIG. 8 is a view illustrating the deviation of a direction in an address state and an impact state of the face side of the club head.

FIG. 9 is a normal distribution diagram illustrating the variation in the face side in the impact state with regard to an advanced learner 1.

FIG. 10 is a normal distribution diagram illustrating the variation in the face side in the impact state with regard to an advanced learner 2.

FIG. 11 is a normal distribution diagram illustrating the variation in the face side in the impact state with regard to an intermediate learner 1.

FIG. 12 is a normal distribution diagram illustrating the variation in the face side in the impact state with regard to an intermediate learner 2.

FIG. 13 is a block diagram illustrating an example of a position computing unit in FIG. 3.

FIG. 14 is a graph illustrating the start of deceleration of a grip bordering at the maximum value of the speed of the grip.

### DESCRIPTION OF EMBODIMENTS

Hereinafter, an embodiment of the invention will be described with reference to the accompanying drawings. Meanwhile, the embodiment, which will be described below, does not unfairly limit the content of the invention which is disclosed in the appended claims, and it is not limited such that all configurations, which are described in the embodiment, are essential as solutions of the invention.

### (1) Swing Analysis System and Swing Analysis Apparatus

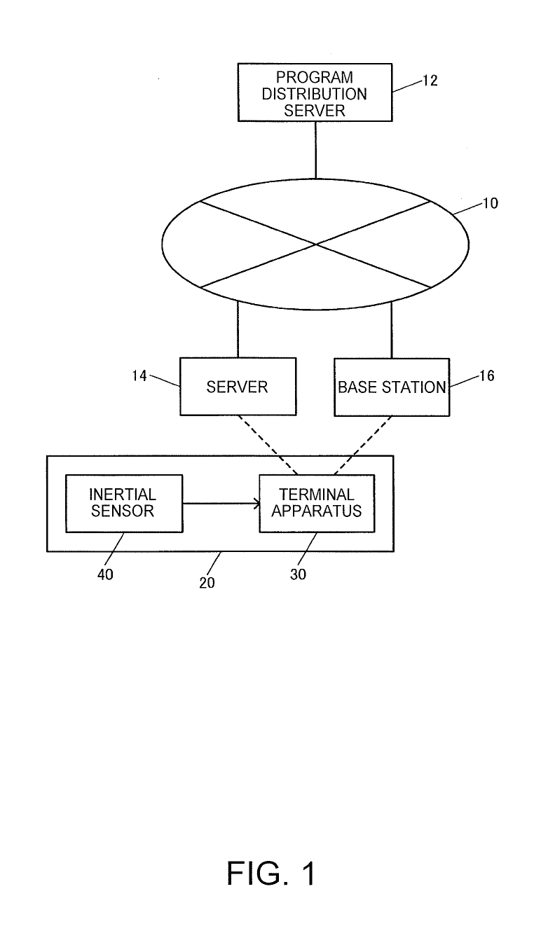

FIG. 1 is a general diagram illustrating a swing analysis system in which the invention is applied for, for example, the supply of a golf swing. As illustrated in FIG. 1, servers 12 and 14, a base station 16, and the like are connected to a network, for example, the Internet 10. The server 12 is a program distribution server which distributes a swing data compression program, a swing analysis program, and the like.

A terminal apparatus 30 includes a mobile terminal, for example, a mobile phone, which can communicate with the program distribution server 12 through the base station 16 and the Internet 10, or a personal computer which can communicate with the program distribution server 12 through the server 14 and the Internet 10. A terminal apparatus 30 and an inertial sensor 40, which is mounted on at least one of an operator (player) who is a detection target and a golf club, forma swing analysis apparatus 20. However, it is possible to call the terminal apparatus 30, which performs swing analysis, the swing analysis apparatus. The swing data compression program and the swing analysis program, which are downloaded from the program distribution server 12, are stored in the storage unit of the terminal apparatus 30.

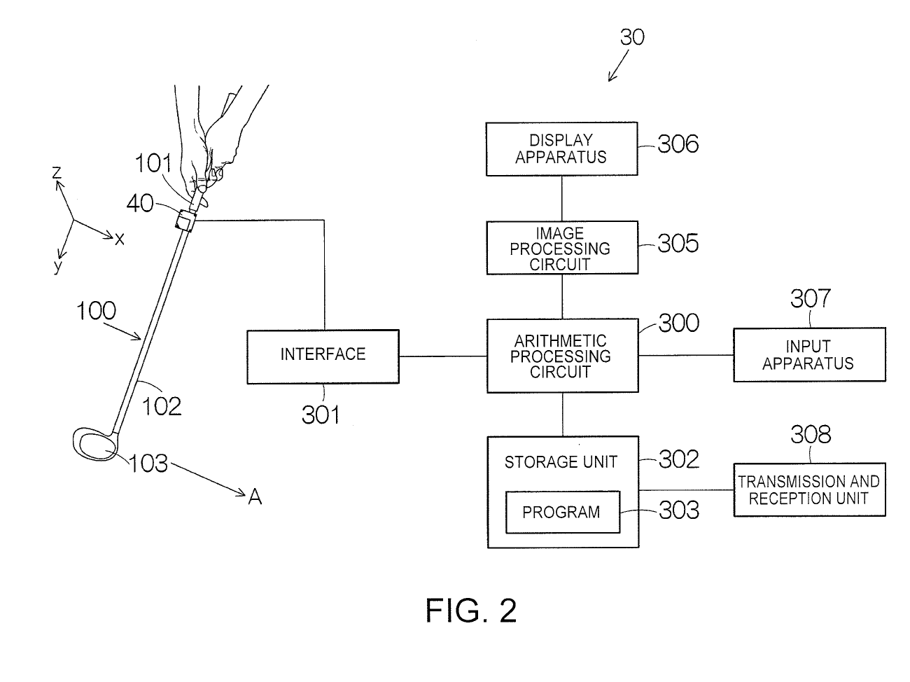

For example, an acceleration sensor and a gyro sensor (angular velocity sensor) are built in the inertial sensor 40. The acceleration sensor can detect respective accelerations in the directions of three axes of x, y, and z which are perpendicular to each other as illustrated in FIG. 2. The gyro sensor can individually detect angular velocities around the respective three axes of x, y, and z which are perpendicular to each other. The inertial sensor 40 outputs detection signals of the acceleration and the angular velocity of the respective axes. The y axis coincides with the axis direction of a shaft 102, and the x axis coincides with a hitting direction A.

The inertial sensor 40 is attached to, for example, a golf club (sports goods) 100, as illustrated in FIG. 2. The golf club 100 includes a grip 101 and the shaft 102. A club head 103 is coupled to the tip of the shaft 102. The inertial sensor 40 is attached to the grip 101 or the shaft 102 of the golf club 100.

The terminal apparatus 30 includes an arithmetic processing circuit 300. The inertial sensor 40 is connected to the arithmetic processing circuit 300 through an interface 301. The interface 301 is connected to the inertial sensor 40 in a wired or wireless manner. A detection signal is supplied to the arithmetic processing circuit 300 from the inertial sensor 40.

A storage unit 302 is connected to the arithmetic processing circuit 300. For example, a program 303, such as the swing data compression program or the swing analysis program, and swing data (data from the inertial sensor 40 or the analysis data thereof) are stored in the storage unit 302. The program 303 is stored after being downloaded from the program distribution server 12. The arithmetic processing circuit 300 implements golf swing analysis by executing the golf swing analysis program. In addition, the arithmetic processing circuit 300 compresses the swing data using the swing data compression program. The storage unit 302 can include a DRAM (Dynamic Random Access Memory), a mass storage apparatus unit, a non-volatile memory, and the like. For example, the above-described program 303 is maintained in the DRAM. When the terminal apparatus 30 includes a mass storage apparatus unit, such as a Hard Disk Drive (HDD) apparatus, it is possible to preserve the program 303 and the data in the HDD. A comparatively small capacity program or data, such as a BIOS (Basic Input/Output System), is stored in the non-volatile memory.

An image processing circuit 305 is connected to the arithmetic processing circuit 300. The arithmetic processing circuit 300 transmits prescribed image data to the image processing circuit 305. A display apparatus 306 is connected to the image processing circuit 305. In connection, a prescribed interface circuit (not shown in the drawing) is connected to the image processing circuit 305. The image processing circuit 305 transmits an image signal to the display apparatus 306 according to input image data. An image which is specified by the image signal is displayed on the screen of the display apparatus 306. A liquid crystal display and other flat panel displays are used as the display apparatus 306. Here, the arithmetic processing circuit 300, the storage unit 302, and the image processing circuit 305 are provided as, for example, a computer apparatus.

An input apparatus 307 is connected to the arithmetic processing circuit 300. The input apparatus 307 includes, for example, alphabet keys and numeric keys. Character information or numeric information is input from the input apparatus 307 to the arithmetic processing circuit 300. In addition, a transmission and reception unit 308 is connected to the storage unit 302. The transmission and reception unit 308 receives a program through the Internet 10 or transmits collected swing data.

### (2) Swing Data Compression Apparatus

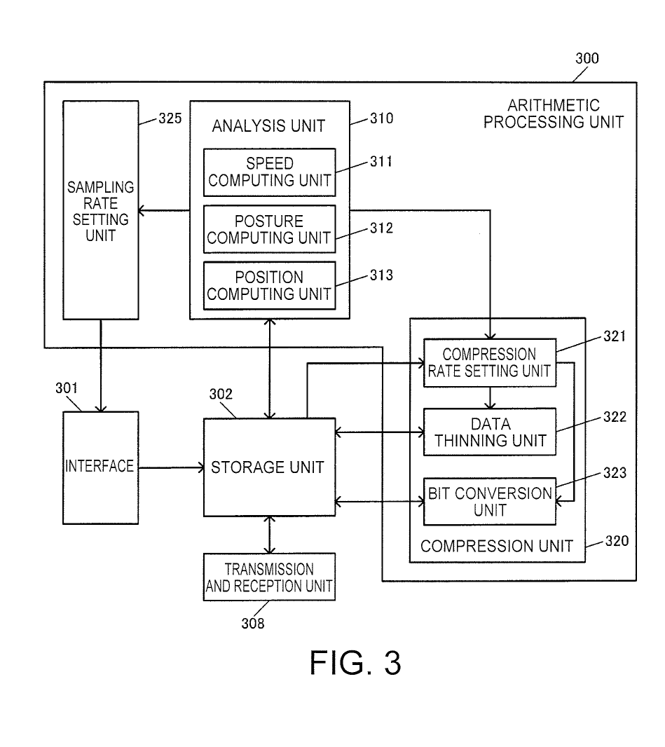

As illustrated in FIG. 3, the arithmetic processing circuit 300 includes an analysis unit 310 which analyzes the swing of the golf club 100 using the swing data stored in the storage unit 302, and a compression unit 320 which compresses the swing data according to a compression rate which is set on the basis of swing analysis results. Meanwhile, in the embodiment, the analysis unit 310 sets the level of a golfer based on the swing analysis results. The level of the golfer is stored in the storage unit 302. The compression unit 320 receives the level from the storage unit 302 or the analysis unit 310, and sets a compression rate according to the level. The arithmetic processing circuit 300 can further include a sampling rate setting unit 325. The sampling rate setting unit 325 variably sets the sampling rate according to the level of the golfer when the inertial sensor 40 collects the swing data.

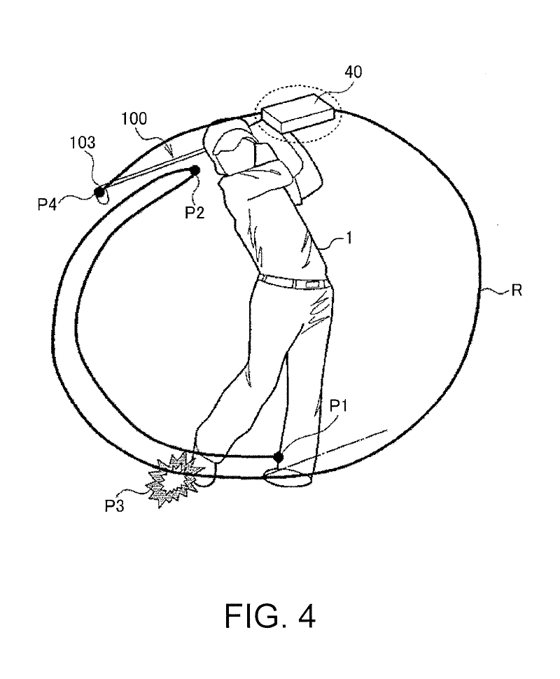

Here, FIG. 4 illustrates the movement trajectory R of one swing of the club head 103 when a golfer 1 swings the golf club 100. The movement trajectory R of one swing includes a swing start (address) position P1, a top position P2, an impact position P3 and a finish position P4.

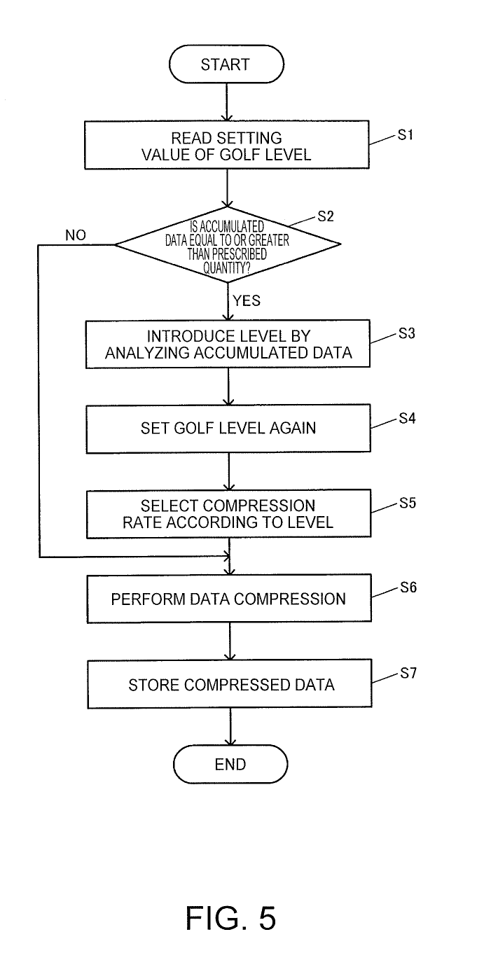

FIG. 5 is a flowchart illustrating the outline of a swing data compression operation. First, the analysis unit 310 reads the level of the golfer 1, which is selected at a previous time from the storage unit 302 (S1). Thereafter, the analysis unit 310 counts swing data, which is accumulated in the storage unit 302, in order to understand the number of swings. When the number of swings is not greater than a threshold (for example, 20 to 30 swings) (No in S2), the compression unit 320 compresses the swing data in the storage unit 302 in accordance with the compression rate according to the past level of the golfer 1 which is read from the storage unit 302 (S6). The reason that the threshold of the number of swings is set is that it is difficult to set a correct level in the process of introducing the level of the golfer (S3) if there is not a fixed or more quantity of data.

If the number of swings is greater than the threshold (YES in S2), the analysis unit 310 analyzes the swing data, which is accumulated in the storage unit 302, and derives the level of the golfer 1 (S3). A level deriving method will be described in detail later. The derived level is updated and preserved in the storage unit 302 (S4). Thereafter, the analysis unit 310 transfers the level to the compression unit 320. The compression unit 320 selects the compression rate according to the received golf level (S5). The compression unit 320 compresses the swing data, which is read from the storage unit 302, in accordance with the compression rate (S6). A compression rate setting method and a compression method will be described in detail later. The compressed swing data is stored in the storage unit 302 (S7). Thereafter, the analysis unit 310 performs various computations, which are necessary for the swing analysis, using the compressed swing data, and generates analysis data. Therefore, it is possible to output analysis results with accuracy at the level of the golfer.

Further, FIG. 3 illustrates examples of the analysis unit 310 and the compression unit 320. The analysis unit 310 includes a speed computing unit 311, a posture computing unit 312, and a position computing unit 313. The speed computing unit 311 computes, for example, the speed of the club head 103, or the like. The posture computing unit 312 computes, for example, the direction (angle) of the face side of the club head 103 in an impact state. The position computing unit 313 computes, for example, the deceleration position of the grip 101 in a case of down swing.

The compression unit 320 can further include a compression rate setting unit 321, which sets the compression rate of the swing data based on the analysis result (level) in the analysis unit 310 or the past level stored in the storage unit 302, a data thinning unit 322 which thins and compresses the swing data according to the set compression rate, and a bit transformation unit 323 which performs transformation of the number of bits which indicates the resolution of the swing data according to the set compression rate. In the embodiment, the compression method is divided into two types. One compression method includes performing compression by thinning all data of swing which continues in time sequence. The other compression method includes performing compression by discarding lower-order bits which form the swing data.

### (3) Data Compression Based on Swing Speed

First, an example in which a compression rate is set based on a speed computed by the speed computing unit 311 of the analysis unit 310 and then data compression is performed will be described. When the speed of the club head 103 is detected, the speed computing unit 311 calculates the acceleration asj (including gravity g) of the club head 103 according to, for example, following expression. When the acceleration is calculated, the speed computing unit 311 specifies the position lsj of the club head 103 according to the unique local coordinate system Σₛ of the inertial sensor 40. The position lsj of the club head 103 can be designated through, for example, the input apparatus 307, and can be stored in the storage unit 302. An acceleration output αs and an angular velocity output ωs are acquired from the inertial sensor 40. An angular acceleration (indicated by attaching a point on the top of the ωs in the following expression) is acquired by differentiating the angular velocity output ωs.

αₛⱼ=αₛ+\dot{ω}ₛ×lₛⱼ+ωₛ×(ωₛ×lₛⱼ)+g [Math. 1]

The speed computing unit 311 calculates the movement speed of the club head 103 based on the calculated acceleration. Here, an integration process is performed on the acceleration at every normal sampling interval dt according to following expression.

$${V}_{sj} ( 0 ) = 0 \\ {V}_{sj} ( t ) = {\sum }_{n = 1}^{t} {α}_{sj} ( n ) \cdot d t \\ ( t = 1 , … , N ) \qquad [ Math . 2 ]$$

When the golf club 100 is a driver, the speed of the club head 103 differs depending on person by as much as twice. An advanced learner swings at a high speed of 50 m/s, and a beginner or a woman swings at a low speed of 30 m/s. In the case of the high-speed swing, the amount of change per unit time is large, and thus the relatively large quantity of data is necessary for analysis. However, in the case of low-speed swing, the amount of change per unit time is small, and thus analysis accuracy is not lowered when the quantity of data is small.

Here, the compression rate setting unit 321 of the compression unit 320 makes setting such that the compression rate of swing data, in which the swing speed is slow, to be higher than the compression rate of the swing data in which the swing speed is rapid (data size acquired before compression/data size acquired after compression). For example, when the compression rate of the swing data, in which a swing speed is equal to or higher than 50 m/s, is set to “1” (that is, non-compression), the compression rate of the swing data, in which a swing speed is 30 m/s, is set to “2” (that is, compression is performed such that the quantity of data becomes half). It is possible to vary data with regard to a swing having a swing speed between 30 m/s to 50 m/s, for example, in stages between the compression rates of “1” and “2”. For example, division is performed into levels 1 to 5. The compression rate of the level 1, which is the uppermost level, is set to “1”, and the compression rate of the level 5, which is the lowermost level, is set to “2”. It is possible to set the compression rate using a table in which the compression rate is primarily acquired from the level.

The data thinning unit 322 performs compression by thinning the swing data in the storage unit 302 according to the compression rate which is set by the compression rate setting unit 321. For example, when the compression rate is “2”, the total quantity of data is compressed into half by thinning one of two swing data sets continuing in time. When the compression rate is “1.5”, one of three swing data sets continuing in time sequence is thinned. When the compression rate is “1.75”, one of four swing data sets continuing in time sequence is thinned. In this manner, it is possible to compress the swing data according to the swing speed. Meanwhile, thinning of one of N (N2) swing data sets continuing in time sequence is called a thinning rate N. In order to increase the compression rate, the thinning rate is lowered. When the data compression is performed, it is possible to reduce the memory capacity of the server 12 or the server 14 to which the swing data is transferred from the terminal apparatus 30. In addition, since it is possible to reduce the quantity of swing data which is transmitted by the transmission and reception unit 308 or it is possible to reduce the total quantity of swing data which is transferred through the Internet 10, it is possible to reduce data transfer time.

In the embodiment, it is possible for the sampling rate setting unit 325 to vary the sampling rate based on the level or the compression rate. The swing data, which is collected in advance, is compressed. However, after the compression rate is set, it is possible for the sampling rate setting unit 325 to set the sampling rate according to the set compression rate. Information related to the set sampling rate is supplied to the inertial sensor 40 through the interface 301. Although the inertial sensor 40 normally uses a fixed sampling rate (for example, 1 kHz), it is possible to lower the sampling rate using a command from the terminal apparatus 30. For example, a sampling rate (for example, 1 kHz) is not changed with regard to the sampling data of a swing speed of 50 m/s or higher. However, in a case of speed which is slower than 50 m/s, the sampling rate is lowered. For example, when the swing speed is 30 m/s or less, the sampling rate is set to 500 Hz. When the swing speed is approximately 40 m/s, the sampling rate is set to 750 Hz. If the sampling rate is lowered, the quantity of swing data which is sampled during one swing is reduced. For example, if the sampling rate is set to 500 kHz, the quantity of data is reduced to half. Accordingly, if the inertial sensor 40 performs sampling according to the changed sampling rate thereafter, compression is not performed by the compression unit 320.

### (4) Data Compression Based on Variation in Posture of Golf Club

The posture computing unit 312 illustrated in FIG. 3 analyzes, for example, the direction (posture) of the face side of the club head 103 in the impact state. According to the results of the analysis, the bit transformation unit 323 of the compression unit 320 compresses the swing data by discarding the lower bits of a plurality of bits which form one swing data set.

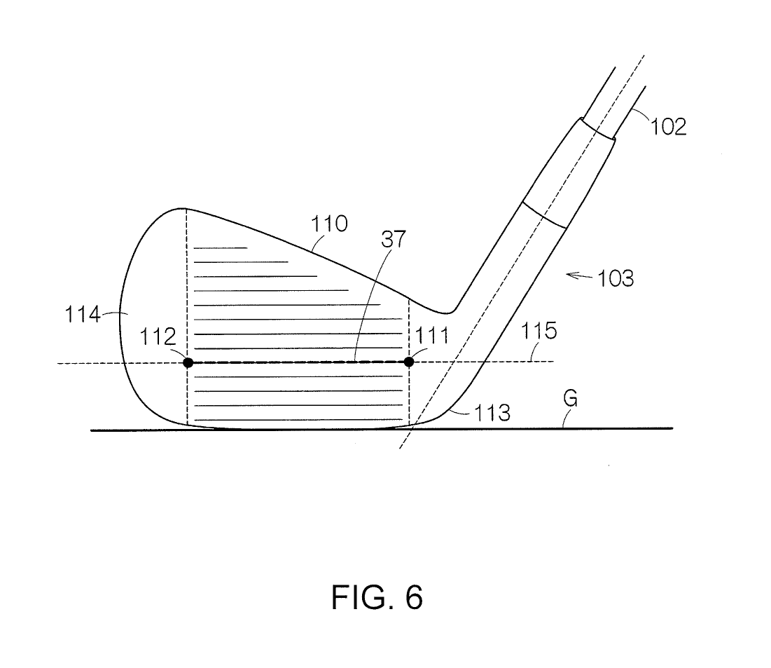

As illustrated in FIG. 6, a first measurement point 111 and a second measurement point 112 are set on q face side 110 of the club head 103. Here, the first measurement point 111 is positioned on the side end of a heel 113 of the face side 110, and the second measurement point 112 is positioned on the side end of a toe 114 of the face side 110. The first measurement point 111 and the second measurement point 112 are allocated on a horizontal plane 115 which is parallel to a ground G. Therefore, it is possible for a line segment 116 which connects the first measurement point 111 with the second measurement point 112 to specify the direction of the face side 110 when being projected into the ground G.

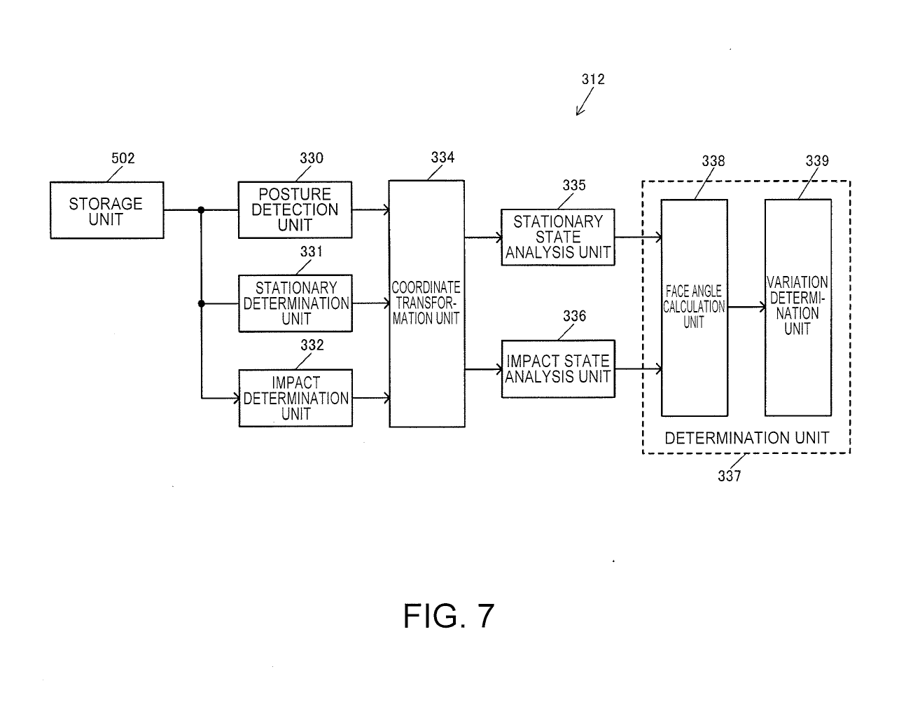

FIG. 7 schematically illustrates the configuration of the posture computing unit 312 according to the embodiment. The posture computing unit 312 includes a posture detection unit 330. The posture detection unit 330 is connected to the storage unit 302. The posture detection unit 330 calculates the posture of the inertial sensor 40 for each sampling point based on the angular velocity around 3 axes. When the calculation is performed, a rotation matrix Rs is specified based on the angular velocity.

$$Rs = ( \begin{matrix}{w}^{2} + {x}^{2} - {y}^{2} - {z}^{2} & 2 ( xy - wz ) & 2 ( xz + wy ) \\ 2 ( xy + wz ) & {w}^{2} - {x}^{2} + {y}^{2} - {z}^{2} & 2 ( yz - wx ) \\ 2 ( xz - wy ) & 2 ( yz + wx ) & {w}^{2} - {x}^{2} - {y}^{2} + {z}^{2}\end{matrix} ) \qquad [ Math . 3 ]$$

Here, when the rotation matrix Rs is specified, quaternion Q is specified.

$$Q = ( w , x , y , z ) \\ w = cos \frac{θ}{2} \\ x = \frac{{ω}_{x}}{\vert \vec{ω} \vert} \cdot sin \frac{θ}{2} \\ y = \frac{{ω}_{y}}{\vert \vec{ω} \vert} \cdot sin \frac{θ}{2} \\ z = \frac{{ω}_{z}}{\vert \vec{ω} \vert} \cdot sin \frac{θ}{2} \qquad [ Math . 4 ]$$

Here, the size of the angular velocity is calculated using the following expression.

|\vec{w}|=ωₓ²+ω_{y}²+ω_{z}² [Math. 5]

However, the measured angular velocity [rad/s] is expressed as the following expression.

\vec{ω}=(ωₓ,ω_{y},ω_{z}) [Math. 6]

A changed angle [rad] per unit time Δt is calculated using the following expression.

θ=|\vec{ω}| [Math. 7]

The posture computing unit 312 includes a stationary determination unit 331 and an impact determination unit 332. The stationary determination unit 331 specifies the stationary state of the golf club 100 based on the output of the storage unit 302. When the stationary determination unit 331 recognizes the stationary state over a prescribed period, the stationary determination unit 331 outputs a stationary notification signal.

The impact determination unit 332 specifies an impact moment based on the output of the storage unit 302. At the impact moment, the output of the inertial sensor 40 is in disorder. When the impact determination unit 332 detects impact, the impact determination unit 332 outputs an impact notification signal.

The posture computing unit 312 includes a coordinate transformation unit 334. Outputs from the posture detection unit 330, the stationary determination unit 331, and the impact determination unit 332 are supplied to the coordinate transformation unit 334. The coordinate transformation unit 334 specifies the posture of the face side 110 of the club head 103 using an absolute reference coordinate system Σ_{xyz} which specifies a real space. When the posture is specified, the coordinate transformation unit 334 specifies the first measurement point 111 and the second measurement point 112 on the face side 31 according to the local coordinate system Σₛ. The coordinate values of the first measurement point 111 and the second measurement point 112 may be stored in, for example, the storage unit 302 in advance. The coordinate transformation unit 334 performs the coordinate transformation on the coordinate values of the local coordinate system Σₛ, and specifies the first measurement point 111 and the second measurement point 112 according to the absolute reference coordinate system Σ_{xyz}. When the coordinate transformation is performed, the coordinate transformation unit 334 specifies the rotation matrix Rs for each sampling point. The change in the posture of the inertial sensor 40 from the start of the measurement corresponds to the integrated value of the rotation matrix Rs from the start of the measurement to the calculation.

The posture computing unit 312 includes a stationary state analysis unit 335 and an impact state analysis unit 336. Outputs from the coordinate transformation unit 334 are supplied to the stationary state analysis unit 335 and the impact state analysis unit 336. The coordinate transformation unit 334 supplies the coordinate value of the first measurement point 111 and the coordinate value of the second measurement point 112, which are acquired after coordinate transformation is performed, to the stationary state analysis unit 335 in accordance with the reception of a stationary notification signal from the stationary state analysis unit 335. In the same manner, the coordinate transformation unit 334 supplies the coordinate value of the first measurement point 111 and the coordinate value of the second measurement point 112, which are acquired after the coordinate transformation is performed, to the impact state analysis unit 336 in accordance with the reception of an impact notification signal from the impact determination unit 332.

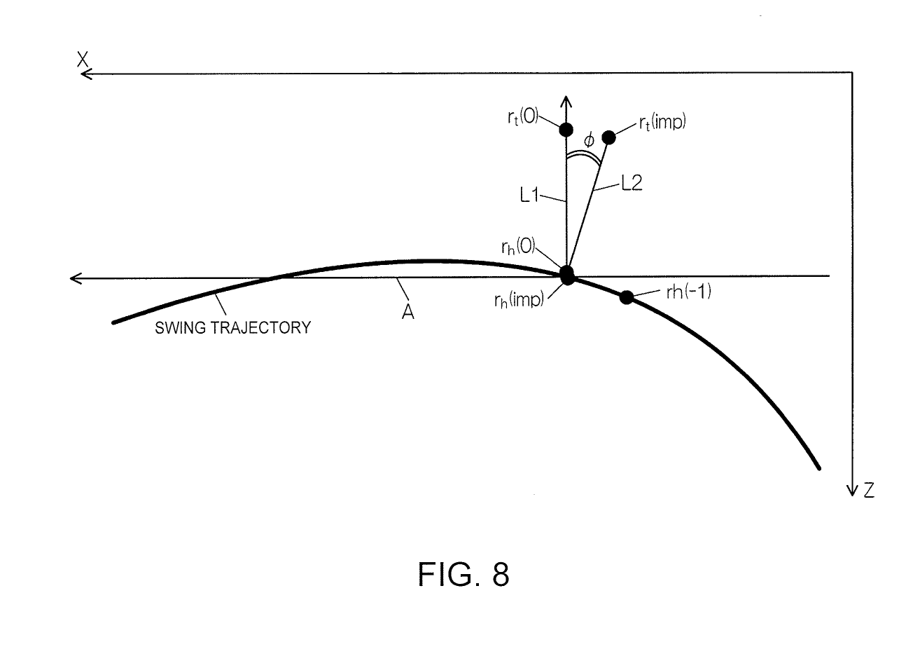

The stationary state analysis unit 335 specifies the posture of the face side 110 using the absolute reference coordinate system Σ_{xyz} in a stationary state (that is, in an address state). When the posture is specified, for example, as shown in FIG. 8, the stationary state analysis unit 335 connects the first measurement point 111=rₕ(0) and the second measurement point 112=rₜ(0) in the stationary state using a first line segment L1 with each other. The posture of the face side 110 is specified using the first line segment L1. At this time, the first line segment L1 is projected on a horizontal plane (surface which extends in parallel to the ground G) which is perpendicular to the y axis in the absolute reference coordinate system Σ_{xyz}.

The impact state analysis unit 336 specifies the posture of the face side 110 using the absolute reference coordinate system Σ_{xyz} in an impact state. When the posture is specified, for example, as shown in FIG. 8, the impact state analysis unit 336 connects the first measurement point 111=rₕ(imp) and the second measurement point 112=rₜ(imp) in the impact state using a second line segment L2. The posture of the face side 110 is specified using the second line segment L2 in the impact state. At this time, as described above, the second line segment L2 is projected on the horizontal plane which is perpendicular to the y axis in the absolute reference coordinate system Σ_{xyz}.

The posture computing unit 312 includes a determination unit 337. The determination unit 337 determines the variation in the face side 110 in the impact state based on information related to the posture of the face side 110. The determination unit 337 includes a face angle calculation unit 338. Outputs of the stationary state analysis unit 335 and the impact state analysis unit 336 are supplied to the face angle calculation unit 338. The face angle calculation unit 338 relatively calculates the angle (face angle) φ of the face side 110 in the impact state with regard to the face side 31 in the stationary state. When the angle φ is calculated, an angle is measured in the horizontal plane of the absolute reference coordinate system Σ_{xyz} between the first line segment L1 and the second line segment L2.

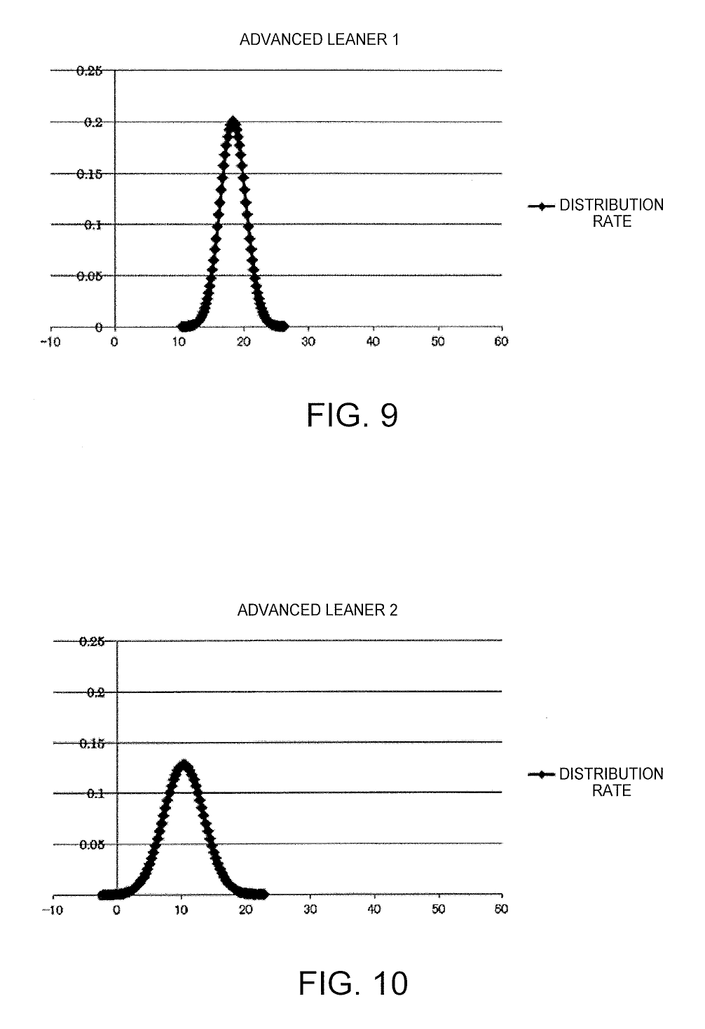

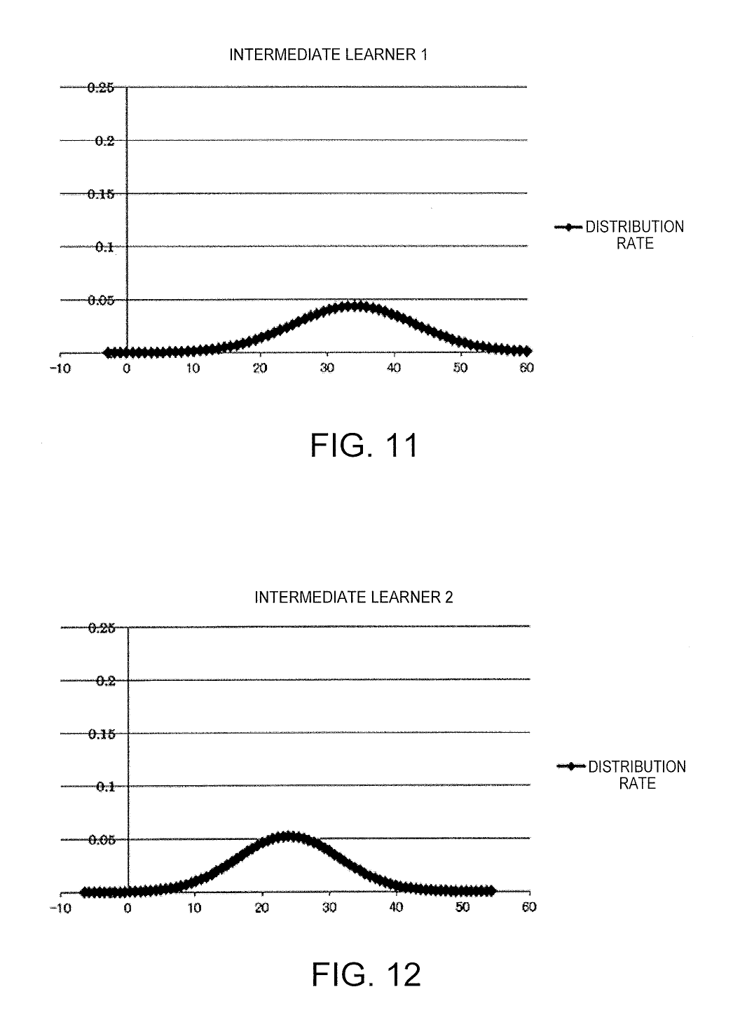

The variation determination unit 339 determines variation in the face angle φ which is output from the face angle calculation unit 338 for each swing. When the variation is determined, it is possible to use standard deviation σ. FIGS. 9 and 10 illustrate the distribution rate of the face angle φ of an advanced learner, and FIGS. 11 and 12 illustrate the distribution rate of the face angle φ of an intermediate learner. Horizontal axes in FIGS. 9 to 12 indicate the face angle φ, and vertical axes indicate the distribution rate.

The standard deviation σ is calculated based on a population. When 100 balls are hit, accuracy is determined on the basis of ±3σ (or ±4σ) in which there is a high possibility that approximately all of the balls are covered. The range of measurement values corresponding to ±3σ is calculated. When a sheet of an advanced learner 1 is shown as an example, a face angle φ of −3σ is 12.33, a face angle φ of +3σ is 24.27, and a width (range) is 11.94 (=24.27-12.33). If accuracy is set such that a difference is distinguished even when 100 balls are equally distributed, 11.94/100=0.1194, and thus accuracy (resolution) in units of 0.1 degree is sufficient (the state is a non-compression state). With regard to an intermediate learner 1, a width of ±3σ is 55.26, 55.26/100=0.5526, and thus accuracy (resolution) in units of 0.5 degrees is sufficient (the state is a compression state).

This means that, the resolution of the intermediate learner may be lower than that of the advanced learner. As above, it is possible to analyze the standard deviation u of the posture of the golf club in multiple swings, and it is possible to make setting such that the compression rate of the swing data of the beginner or the intermediate learner, in which a range of ±3σ or ±4σ is wide, is higher than the compression rate of the swing data of the advanced learner in which a range of ±3σ or ±4σ is narrow.

The variation determination unit 339 acquires the standard deviation σ of the face angle φ, and acquires a range of ±3σ or ±4σ. The acquired range of ±3σ or ±4σ is output as, for example, one of a 5-stage level.

The level is input to the compression rate setting unit 321 of the compression unit 320. The compression rate setting unit 321 sets the number of digits of lower bits which are discarded by the bit transformation unit 323. The bit transformation unit 323 discards the lower bits from bits, which form one swing data set, corresponding to the number of digits which is set by the compression rate setting unit 321. For example, in order to perform data compression at accuracy (resolution) in units of 0.5 degrees by compressing N-digit binary data of the non-compression data in which accuracy (resolution) of the face angle φ in units of 0.1 degrees is acquired, two lower digits may be discarded. Meanwhile, it is possible to thin the data by collectively discarding the lower bit digits, and thus it is possible to further increase the compression rate.

### (5) Data Compression Based on Variation in Position of Golf Club

The position computing unit 313 illustrated in FIG. 3 analyzes, for example, the deceleration position of the grip 101 when down swing is performed. According to the result of analysis, the bit transformation unit 323 of the compression unit 320 discards the lower bits of a plurality of bits which form one swing data set, and compresses the swing data.

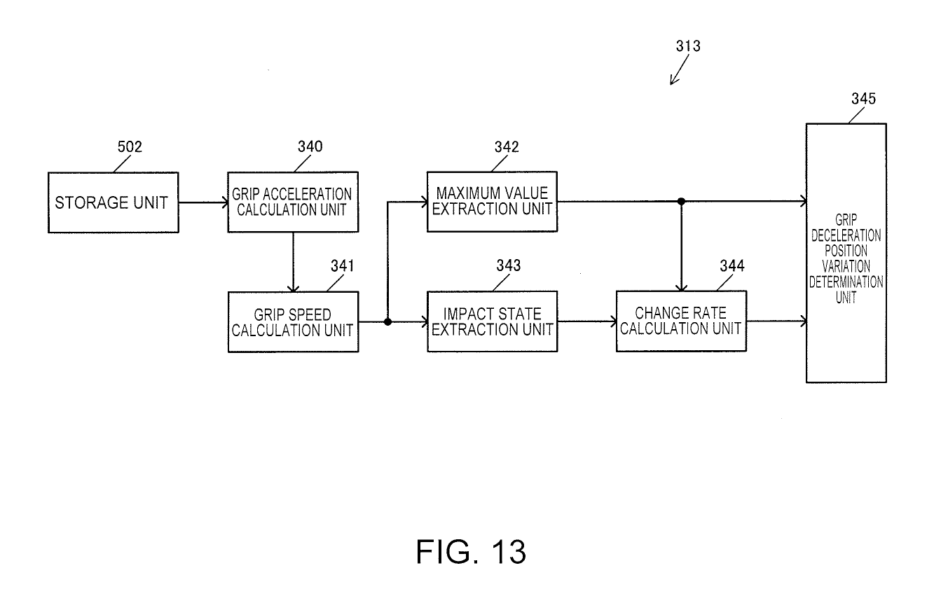

In FIG. 13 which illustrates an example of the position computing unit 313 illustrated in FIG. 3, a grip acceleration calculation unit 340 calculates the acceleration ash of the grip 101 using a length lsh from the inertial sensor 40 to the grip 101 instead of the length lsj of the club head 103 in the above-described Math. 1 based on the swing data stored in a storage unit 502. The grip speed calculation unit 341 calculates the speed Vsh(t) of the grip 101 using Vsh(0)=0 and the acceleration ash of the grip 101, which is acquired by the grip acceleration calculation unit 340, in the above-described Math. 2.

A maximum value extraction unit 342 extracts the maximum value of the movement speed of the grip 101. An impact state extraction unit 343 extracts the movement speed of the grip 101 in an impact state. A change rate calculation unit 344 calculates a change rate η of a speed based on the size of the movement speed which is extracted by the maximum value extraction unit 342 and the impact state extraction unit 343 according to the following expression.

$$η = 1 - \frac{Speed in impact state}{Maximum value of speed} \qquad [ Math . 8 ]$$

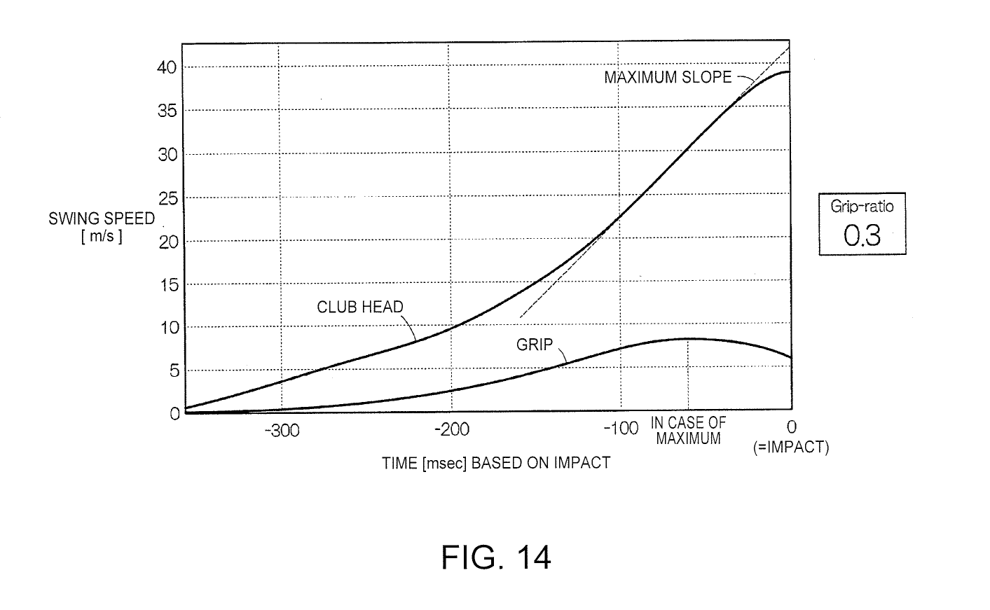

Here, FIG. 14 illustrates the change in the speed of the grip 101 when the change rate η of the speed is “0.3”. Meanwhile, FIG. 14 illustrates the change in the speed of the club head 103 for reference. According to a time axis of impact, the movement of the grip 101 is decelerated from the maximum value of the movement speed. Therefore, if the position (time) of the maximum value K of the movement speed is specified, the grip 101 is decelerated at the position of the maximum value, and thus the deceleration position of the grip is specified. If the movement speed of the grip 13b is not decelerated, the movement speed in the impact state corresponds to the maximum value of the movement speed without changing. As a result, the change rate becomes “0 (zero)”, and thus the grip 101 is not decelerated during the down swing. When the change rate η of the speed is “0”, data compression in which the deceleration position of the grip 101 is used as an index is not performed. When the change rate 11 of the speed is not “0”, the data compression in which the deceleration position of the grip 101 is used as an index is performed.

A grip deceleration position variation determination unit 345, which is connected to the maximum value extraction unit 342 and the change rate calculation unit 344, determines the variation in a grip speed deceleration position (=the position of the maximum value) for each swing when the change rate η of the speed is not “0”. When the variation is determined, it is possible to use the standard deviation σ similarly to the variation determination unit 339. The grip deceleration position variation determination unit 345 acquires the standard deviation σ of the deceleration position of the grip, and further acquires the range of ±3σ or ±4σ. The acquired range of ±3σ or ±4σ is output as, for example, one level of 5 stages.

The level is input to the compression rate setting unit 321 of the compression unit 320. The compression rate setting unit 321 sets the number of digits of the lower bits which are discarded by the bit transformation unit 323. The bit transformation unit 323 discards lower bits, corresponding to the number of digits which is set by the compression rate setting unit 321, from the bits which form one swing data set. In this case, it is possible to collectively discard lower bit digits and thin the data, and thus it is possible to further increase the compression rate.

Although the embodiment has been described in detail as above, those skilled in the art can easily understand that various modifications are possible without substantially departing from the new matters and advantages of the invention. Therefore, all modification examples are included in the scope of the invention. For example, in the specification and the drawings, terms described at least once with different terms in a broad sense or synonymous meaning can be replaced with the different terms in any part of the specification and the drawings.

For example, the analysis unit 310 illustrated in FIG. 3 can be commonly used as an analysis unit, which performs original swing analysis using swing data acquired after compression in addition to swing analysis in order to set the compression rate. The analysis unit 310 can generate swing analysis data, such as the swing trajectory, based on the compressed swing data, and can store the swing analysis data in the storage unit 502.

In addition, the example of computation performed by the speed computing unit 311, the posture computing unit 312, or the position computing unit 313, which is provided in the analysis unit 310 illustrated in FIG. 3, is an example, and computation may be performed with regard to other points. For example, the position computing unit 313 may compute the height of a transition position (top position) P2 illustrated in FIG. 4, or the width of swing between positions P2 and P4. When compared with a full shot, the swing speed of a half shot is slow. Accordingly, data may be thinned based on the height of the position P2 or the width of a swing between the positions P2 and P4 instead of the swing speed.

Meanwhile, the invention is not limited to golf, and can be widely applied to sports, such as tennis, table tennis, badminton, or baseball, in which sporting goods are swung, and a swing operation in addition to the sport.

### REFERENCE SIGNS LIST

1: golfer
30: terminal apparatus
40: inertial sensor
100: golf club
101: grip
102: shaft
103: club head
300: arithmetic processing circuit
302: storage unit
310: analysis unit
320: compression unit
321: compression rate setting unit
322: data thinning unit
323: bit transformation unit

1. A swing data compression method comprising:
storing swing data which is output from an inertial sensor mounted on a detection target;
analyzing a swing of the detection target using the stored swing data; and
compressing the swing data according to a compression rate which is set based on a result of swing analysis.

2. The swing data compression method according to claim 1, further comprising:
analyzing a swing speed of the detection target; and
making setting such that the compression rate of the swing data in which the swing speed is slow is higher than the compression rate of the swing data in which the swing speed is rapid.

3. The swing data compression method according to claim 2, further comprising:
compressing the swing data by thinning the swing data of one swing which continues in time sequence according to the compression rate.

4. The swing data compression method according to claim 1, further comprising:
analyzing at least one of posture and position of the detection target in multiple swings; and
making setting such that the compression rate of the swing data, in which variation in at least one of the posture and the position of the detection target in each swing is large, is higher than the compression rate of the swing data in which variation in at least one of the posture and the position of the detection target in each swing is small.

5. The swing data compression method according to claim 4, further comprising:
compressing the swing data by discarding lower bits of a plurality of bits which form one swing data set according to the compression rate.

6. The swing data compression method according to claim 4, further comprising:
analyzing a standard deviation σ of at least one of the posture and the position of the detection target in the multiple swings; and
making setting such that the compression rate of the swing data, in which a range of ±3σ or ±4σ is wide, is higher than the compression rate of the swing data, in which the range of ±3σ or ±4σ is narrow.

7. A swing data compression apparatus comprising:
a storage unit that stores swing data which is output from an inertial sensor mounted on a detection target;
an analysis unit that analyzes a swing of the detection target using the swing data stored in the storage unit; and
a compression unit that compresses the swing data according to a compression rate which is set based on a result of swing analysis.

8. A swing analysis apparatus comprising:
a storage unit that stores swing data which is output from an inertial sensor mounted on a detection target;
an analysis unit that analyzes a swing of the detection target using the swing data stored in the storage unit; and
a compression unit that compresses the swing data according to a compression rate which is set based on a result of swing analysis.

9. The swing analysis apparatus according to claim 8, further comprising:
a sampling rate setting unit that sets a sampling rate such that the inertial sensor performs sampling on the swing data based on the swing analysis.

10. A swing data compression program causing a computer to perform:
a procedure for storing swing data which is output from an inertial sensor mounted on a detection target;
a procedure for analyzing a swing of the detection target using the stored swing data; and
a procedure for compressing the swing data according to a compression rate which is set based on a result of swing analysis.

11. The swing data compression method according to claim 5, further comprising:
analyzing a standard deviation σ of at least one of the posture and the position of the detection target in the multiple swings; and
making setting such that the compression rate of the swing data, in which a range of ±3σ or ±4σ is wide, is higher than the compression rate of the swing data, in which the range of ±3σ or ±4σ is narrow.
